(19) United States
(12) Patent Application Publication
(10) Pub. No.: US 2012/0116178 A1
(43) Pub. Date: May 10, 2012

(54) VITAL SIGN SENSING SYSTEM, VITAL SIGN OBTAINING DEVICE, AND DISPOSABLE DEVICE

(75) Inventors: YI-HSIN HUANG, New Taipei City (TW); Chin-Feng Lai, New Taipei City (TW); Chao-Wang Chen, New Taipei City (TW)

(73) Assignee: TAIDOC TECHNOLOGY CORPORAT

(21) Appl. No.: 13/291,963

(22) Filed: Nov. 8, 2011

(30) Foreign Application Priority Data
Nov. 10, 2010 (TW) 099138752

(51) Int. Cl. A61B 5/00

(52) U.S. Cl. 600/300

(57) ABSTRACT

The present invention related to a vital sign sensing system, a vital sign obtaining device, and a disposable device. The vital sign sensing system includes the vital sign obtaining device and the disposable device. The disposable device includes an elasticity stopper and a movable fastener. The vital sign obtaining device includes a protrusion. The movable fastener is assembled on the elasticity stopper so that the elasticity stopper is showed a flexion state. In a first procedure that the disposable device is assembled to the vital sign obtaining device, the movable fastener is uninstalled from the elasticity stopper by the protrusion, and the elasticity stopper is converted from the flexion state to an expansion state so as to prevent the disposable device from being reused in vital sign obtaining device.

### BACKGROUND OF THE INVENTION

1. Field of the Invention

The present invention relates to a disposable device of a vital sign sensing system, and especially is relevant to a technology for preventing repeatedly used of the disposable device.

2. Description of the Related Art

With the progress of medical device industry, blood glucose measuring device and electronic pregnancy diagnosis device for use at home have improved. The users can measure blood glucose or detect if pregnant at home by themselves.

Generally speaking, the blood glucose measuring device must cooperate with the blood glucose test strip for use. The electronic pregnancy diagnosis device also needs to collocate with the pregnancy test strip for use. These test strips are disposable and can not be repeatedly used. In other words, the users take used disposable test strip for use again will cause a wrong determination of the device. The wrong determination may cause great damage.

For example, the pregnant woman assumes that she is not pregnant by the wrong determination so she eats the harmful food or drug may cause the fetal deformity even miscarriage. Thus, a need exists for improved the device such as vital sign sensing system, vital sign obtaining device, and disposable device.

### SUMMARY OF THE INVENTION

According to one aspect of the present invention is to provide a vital sign sensing system for reducing the probability of a wrong determination, a disposable device for preventing repeatedly used, and a vital sign obtaining device for resisting the used disposable device to be used again.

In one aspect of the present invention, the present invention provides a vital sign sensing system, including a disposable device and a vital sign obtaining device. The disposable device may include a first assembly, an elasticity stopper, a movable fastener and a detective region. The elasticity stopper is formed on the first assembly, which will be in a flexion state and an expansion state. When in nature, the elasticity stopper is in the expansion state. The movable fastener is movably assembled on the elasticity stopper to limit the elasticity stopper in the flexion state. The detective region is disposed on the first assembly. The vital sign obtaining device may include a second assembly, a sensing module and at least one protrusion. The sensing module is disposed on the second assembly, which is used for obtaining vital sign from the detective region. The protrusion described above is preferably formed on the second assembly. In a first time that the first assembly is assembled to the second assembly, the movable fastener is uninstalled from the elasticity stopper by the push of the protrusion. In addition, after the first assembly uninstalled from the second assembly, the elasticity stopper is converted from the flexion state to the expansion state so as to prevent the first assembly from being reassembled with the second assembly.

In an embodiment in accordance with the present invention, the elasticity stopper may include at least one elasticity arm. The elasticity arm extends out from the first assembly. An exterior of the elasticity arm has a cavity part. In the expansion state, the cavity part clasps the protrusion so as to prevent the first assembly being reassembled with the second assembly.

In an embodiment in accordance with the present invention, the disposable device may further include a hook and a track. The hook is formed in the first assembly. The track is formed in the first assembly between the hook and the elasticity stopper. In a first time that the first assembly is assembled to the second assembly, the movable fastener uninstalls from the elasticity stopper to moving along with the track, and clasping the hook for each other.

In an embodiment in accordance with the present invention, the elasticity stopper may have an indicative symbol. When the movable fastener is assembled to the elasticity stopper, the indicative symbol has been hidden. When the movable fastener is uninstalled from the elasticity stopper, the indicative symbol has been showed.

In another aspect in accordance with the present invention, a vital sign sensing system including a disposable device as described above is provided.

In one another aspect in accordance with the present invention, a vital sign sensing system including a vital sign obtaining device as described above is provided.

According to the aspect of the present invention as description above, the disposable device is disposed the elasticity stopper thereon. After the disposable device has been used, the elasticity stopper converts the state which to preventing the disposable device to be used again. As a result, it can reduce the probability of the vital sign obtaining device having the wrong determination.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is an exploded perspective view of an embodiment of a vital sign sensing system in accordance with the present invention;

FIG. 2 is a schematic perspective view of an embodiment of a movable fastener in accordance with the present invention;

FIGS. 3A to 3C are schematic perspective views of an embodiment of an elasticity stopper in a flexion state from different side views in accordance with the present invention;

FIGS. 4A to 4C are schematic perspective diagrams showing views of the elasticity stopper in the embodiment of FIG. 3 in an expansion state from different side views in accordance with the present invention;

FIG. 5 is a perspective view of an embodiment of a disposable device assembled on a vital sign obtaining device in accordance with the present invention; and

FIGS. 6 to 8 are partial cross-sectional views of the disposable device in the embodiment of FIG. 5 assembled on the vital sign obtaining device in accordance with the present invention.

### DESCRIPTION OF THE PREFERRED EMBODIMENTS

For the purposes of promoting an understanding of the principles of the invention, reference will now be made to the embodiment illustrated in the drawings, and specific language will be used to describe that embodiment. It will nevertheless be understood that no limitation of the scope of the invention is intended. Alterations and modifications in the illustrated device, and further applications of the principles of the invention as illustrated therein, as would normally occur to one skilled in the art to which the invention relates are contemplated, are desired to be protected. Such alternative embodiments require certain adaptations to the embodiments discussed herein that would be obvious to those skilled in the art.

The following description and accompanying drawings are some examples in accordance with the present invention. The same symbol herein in the drawings indicates the same or similar structure.

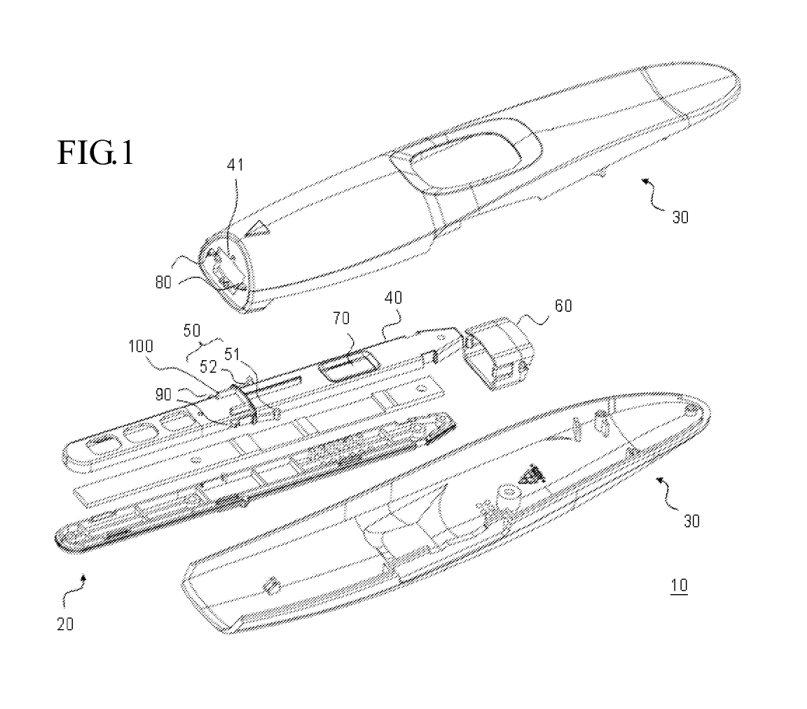

With reference to FIG. 1, it is an exploded perspective view of an embodiment of a vital sign sensing system in accordance with the present invention. The vital sign sensing system (10) comprises a disposable device (20) and a vital sign obtaining device (30). The disposable device (20) includes a pregnancy test strip and a housing. The pregnancy test strip can be used in reaction with the specimen and showed whether there is pregnancy. Generally speaking, when the pregnancy test strip is showed a result of one line in a control line, it means no pregnancy. When the pregnancy test strip is showed a result of two lines in the control line and a test line, it means pregnancy. In the other embodiments, those skilled in the art can change the showed form of the result of the pregnancy test strip.

To be noticed, users who never used the pregnancy test strip do not know the meaning of the result showed in one line or two lines. Additionally, the test line could not be apparent in early pregnancy. Therefore, in the present embodiment, the vital sign obtaining device (30) can assist with identified the result of pregnancy test strip, so as to prevent a wrong determination by the users.

In the present embodiment, the housing is not only protecting the pregnancy test strip but also helping the disposable device (20) assembled in the vital sign obtaining device (30). In the present embodiment, the housing is improved in order to prevent the disposable device (20) for repeatedly used, so that the used disposable device (20) can not be assembled to the vital sign obtaining device (30) again. As a result, it can prevent the device from making the wrong determination.

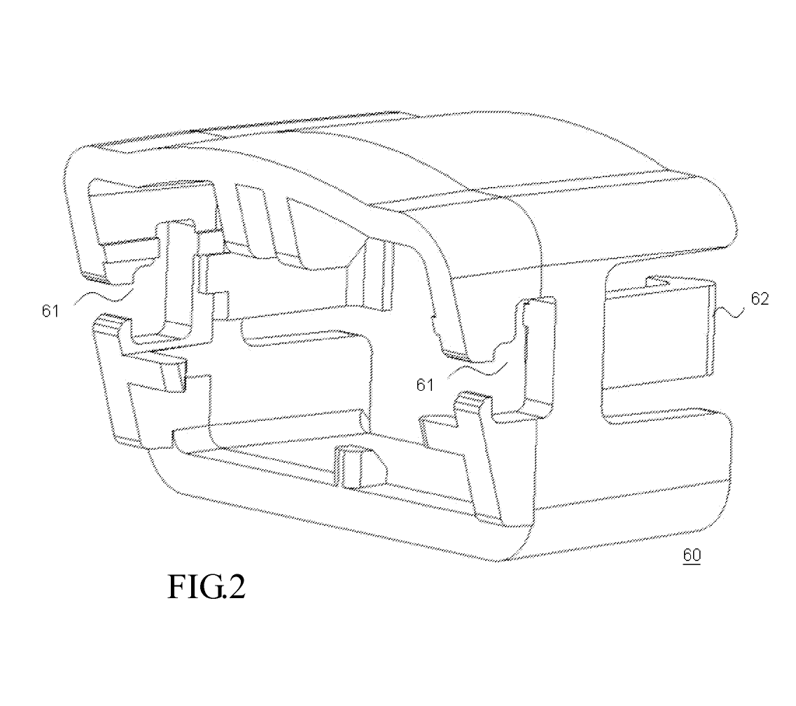

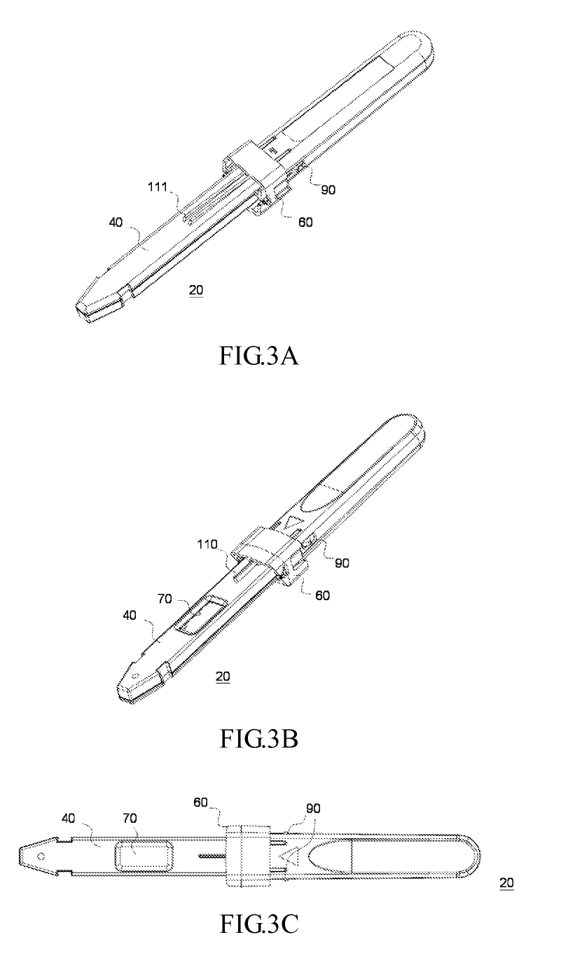

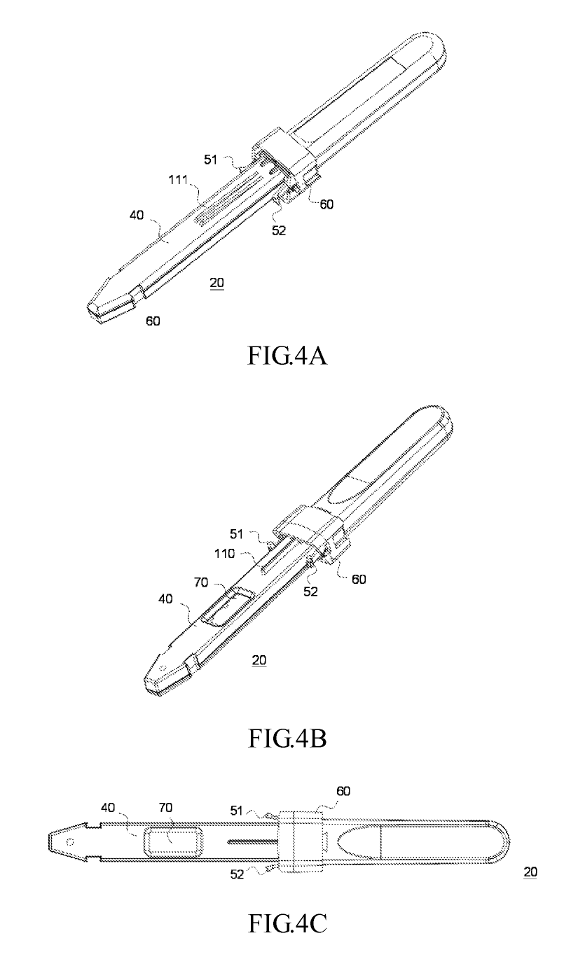

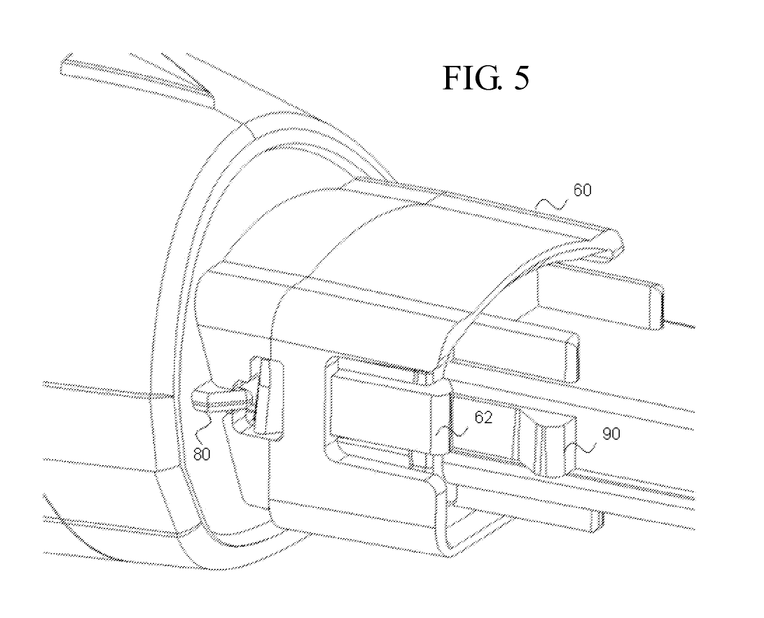

FIG. 2 is a schematic perspective view of an embodiment of a movable fastener in accordance with the present invention. FIGS. 3A to 3C are schematic perspective views of an embodiment of an elasticity stopper in a flexion state from different side views in accordance with the present invention. FIGS. 4A to 4C are schematic perspective views of the embodiment of the elasticity stopper in FIG. 3 in an expansion state in accordance with the present invention. FIG. 5 is a perspective view of an embodiment of a disposable device assembled on the vital sign obtaining device in accordance with the present invention. Further referring to FIGS. 1 to 5 in combination, the disposable device (20) includes an assembly (40), an elasticity stopper (50), a movable fastener (60), and a detective region (70). The elasticity stopper (50) is formed on the assembly (40). The elasticity stopper (50) includes at least one elasticity arm. The embodiment illustrates two elasticity arms (51, 52) for instance. The elasticity arms (51, 52) are respectively disposed on and extended from the two lateral sides of the assembly (40). The elasticity arms (51, 52) could be in a flexion state (as FIG. 3 shown) and in an expansion state (as FIG. 4 shown). The elasticity arms (51, 52) are in the expansion state when they are unforced.

Following the description above, the movable fastener (60) is assembled on the assembly (40) and located out of the elasticity stopper (50) so press the elasticity arms (51, 52) in the flexion state. The movable fastener (60) includes a fixture groove (61) and a hook (62) (as FIG. 2 shown). Preferably, the movable fastener (60) comprises two fixture grooves (61) and two hooks (62) respectively disposed on two sides of the movable fastener (60) in the present embodiment. The elasticity arms (51, 52) hook in the fixture grooves (61) respectively for preventing the movable fastener (60) from shifting by the users.

In addition, the disposable device (20) further includes a second hook (90), a track (100) (as FIG. 1 shown) and an error-proof device. In the present embodiment, the disposable device (20) includes two error-proof devices (110, 111). The second hook (90) is used to cooperate with the hook (62). Preferably, the disposable device (20) comprises two second hooks (90) which are respectively formed on the two lateral sides of the assembly (40). The hook (62) clasps the second hook (90) for each other (as FIG. 5 shown). The track (100) is formed on the assembly (40), which could be located between the second hook (90) and the elasticity stopper (50) for instance. The disposable device (20) can slide and follow with the track (100). The error-proof device (110, 111) can prevent the disposable device (20) from being assembled on the vital sign obtaining device (30) by the wrong direction. To be noticed, the error-proof device (110, 111) also can be the track for the movable fastener (60) sliding on.

The detective region (70) is disposed on the assembly (40). The result of the pregnancy test strip is showed on the detective region (70).

The vital sign obtaining device (30) includes a second assembly (41), a sensing module (not shown) and at least one protrusion (80). The sensing module can be disposed in the second assembly (41), which obtaining the vital sign from the detective region (70). For example, the sensing module can be the reflective optical sensing module. The reflective optical sensing module, for instance, can compose of one or more light emitting diode and photo diode. The light emitting diode can illuminate the detective region (70) and the photo diode can detect the reflective light from the detective region (70) to let the vital sign obtaining device (30) determine whether there is pregnancy. In the different embodiment, the sensing module can change to other type corresponding to the form of the disposable device or the demand of the users. The protrusion (80) is formed on the assembly (41) and used to uninstall the movable fastener (60). Additionally, the protrusion (80) also can resist the elasticity stopper (50) in the expansion state.

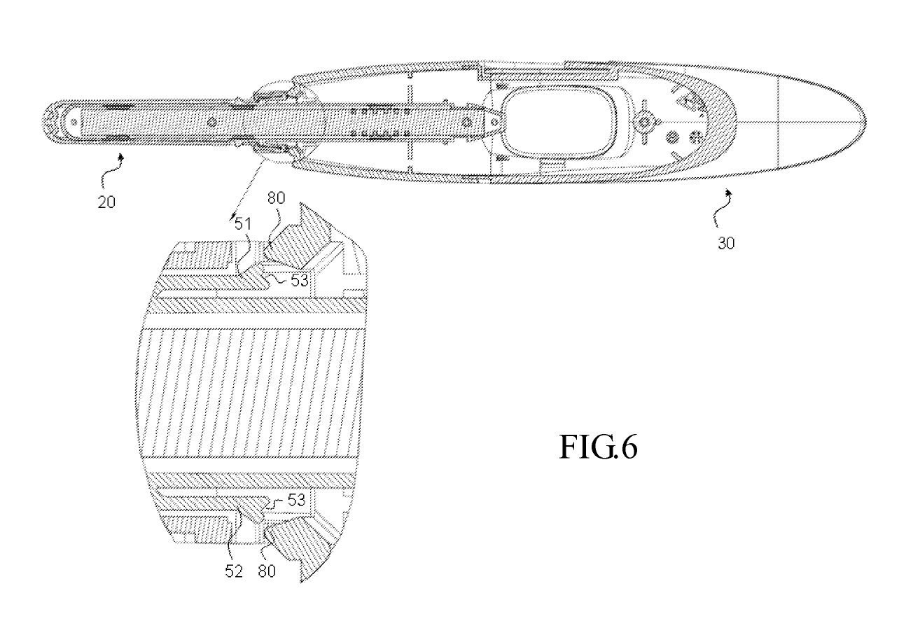

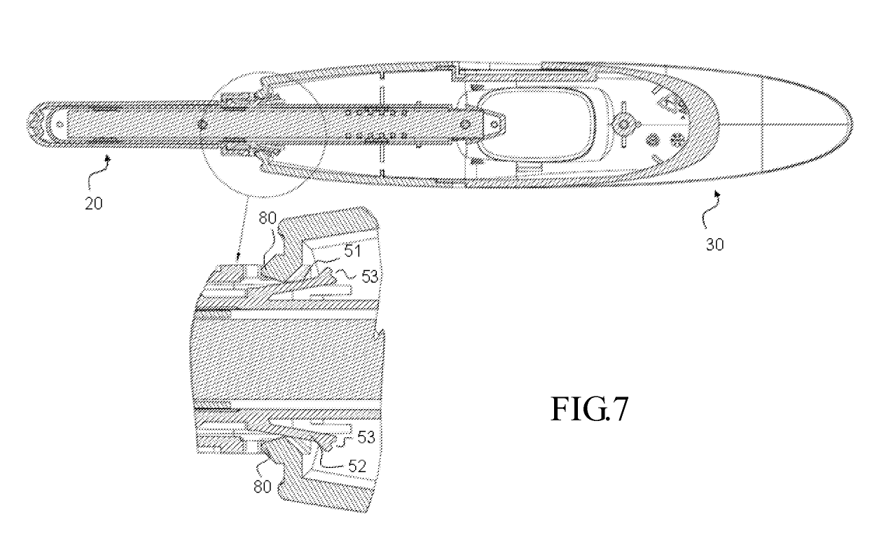

FIGS. 6 to 8 are partial cross-sectional views of the disposable device assembled on the vital sign obtaining device in accordance with the present invention. Further referring to FIGS. 1 and 6 to 8 in combination, in a first procedure that the assembly (40) is assembling to the second assembly (41) (as FIG. 6 shown), the movable fastener (60) is uninstalled from the elasticity stopper (50) by the protrusion (80) (as FIG. 7 shown), and the movable fastener (60) can slide on the track (100) so as to clasp with the second hook (90) for each other.

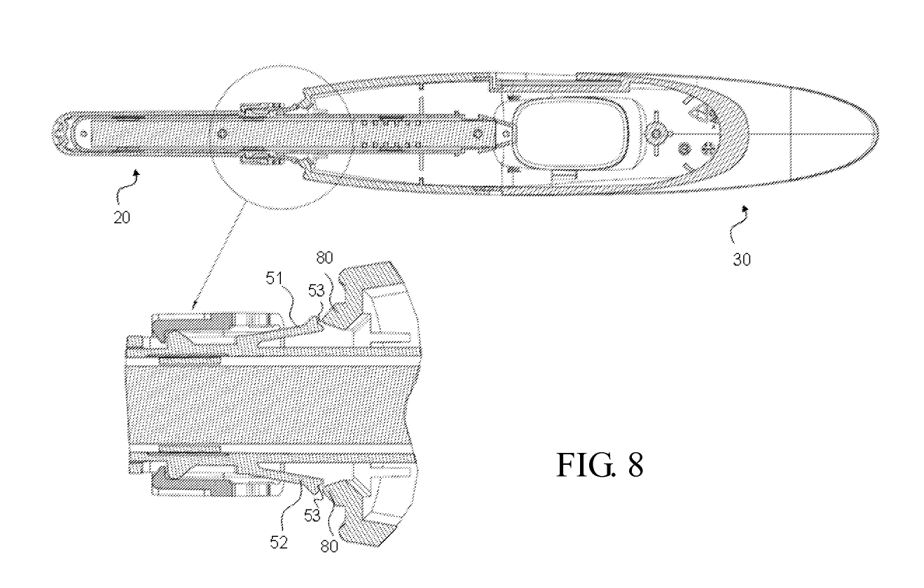

After the sensing module of the vital sign obtaining device (30) obtains the vital sign from the detective region (70), the disposable device (20) ejects from the vital sign obtaining device (30). To be noticed, after the assembly (40) uninstalled from the second assembly (41), the elasticity stopper (50) is converted from the flexion state to an expansion state (as FIG. 8 shown) so as to prevent the assembly (40) from being assembled with the second assembly (41) again. When the users want to assemble the used disposable device (20) to the vital sign obtaining device (30) again, the protrusion (80) resists the elasticity arms (51, 52) (as FIG. 8 shown). As a result, it can prevent repeatedly from using the used disposable device (20) so as to improve the question of the device making a wrong determination and reducing the risk on the users. In the present embodiment, after the movable fastener (60) clasped the second hook (90) for each other, the movable fastener (60) is not easy to move again. The advantage is preventing the movable fastener (60) from installing with the elasticity stopper (50) by the users so as that the elasticity stopper (50) converted to the flexion state again. In addition, it can prevent the movable fastener (60) from falling from the disposable device (20) so as to avoid the users mistaking the malfunction of the disposable device (20).

Additionally, in the present embodiment, an exterior of the elasticity arms (51, 52) respectively have a cavity part (53). In an expansion state, the cavity part (53) clasps the protrusion (80) so as to prevent the assembly (40) assembling with the second assembly (41) again. The advantage of disposed the cavity part (53) on the elasticity arms (51, 52) is the elasticity arms (51, 52) hard be forced to be bending when the cavity part (53) clasped the protrusion (80) for each other.

Besides, in the present embodiment, the elasticity stopper (50) could be designed with an indicative symbol. The indicative symbol could be a notable color or mark. For example, there can be the red color or the mark of “X”, “USED” and so on, but shall not be limited with it. When the movable fastener (60) is assembled to the elasticity stopper (50), the indicative symbol has been hidden. The users can not see the indicative symbol from the outside of the disposable device (20). On the other hand, when the movable fastener (60) is uninstalled to the elasticity stopper (50), the indicative symbol has been shown to indicate the disposable device (20) had been used. The users can easily notice the indicative symbol from the outside of the disposable device (20).

To be noticed, although possible types of the vital sign sensing system, the vital sign obtaining device and the disposable device in accordance with the present invention has been described in the embodiment above, those skilled in the art shall recognized that the vital sign sensing system, the vital sign obtaining device and the disposable device can be designed differently. Therefore, the spirit of the present invention shall not be limited to these possible types of the vital sign sensing system, the vital sign obtaining device and the disposable device in accordance with the present invention. In other words, the process of the disposable device assembled to the other device which the structure getting conformation change so that the disposable device can not be repeatedly used is the key spirit and scope of the present invention. The followings are some other embodiments in accordance with the present invention for those skilled in the art to know more about the spirit of the present invention.

In the above embodiment of FIG. 1, although the elasticity stopper (50) employed in the embodiment described as two elasticity arms (51, 52) for instance, but the present invention shall not be limited with it. In the other embodiment, the elasticity stopper (50) can comprise one or three above of the elasticity arm. The number of protrusion (80) can adjust corresponding to the elasticity arm. In addition, the elasticity stopper (50) does not limit to dispose on the two sides of the disposable device (20). Those skilled in the art can change the configuration type of the elasticity stopper (50) as needed. For example, in the other embodiment, the elasticity stopper (50) can distribute circularly around the disposable device (20).

In the above embodiment of FIG. 1, the track (100) is one example of the choice. In the other embodiment, it can adopt different number or length of the track. Additionally, in the other embodiment, some part of the track (100) can dispose beyond the second hook (90) and the elasticity stopper (50).

In the above embodiment of FIG. 1, the protrusion (80) is not only used to uninstall the movable fastener (60) but also resist the elasticity stopper (50) to be in the expansion state. But that is one choice of the embodiment. In the other embodiment, it can use different structure to perform the function described above respectively.

In the above embodiment of FIG. 8, the exterior of the elasticity arms (51, 52) for matching with the conformation of the protrusion (80) form the two talon-shaped structures (the cavity part 53). However, the conformation of the structure is one choice of the embodiment, and the present invention shall not be limited with it. For example, the protrusion (80) can change to be the pestle type, and the exterior of the elasticity arms (51, 52) are corresponding changed to be the mortar type.

The above described technology of preventing disposable device from repeatedly being used can apply for the other vital sign sensing system such as for blood glucose or ovulation determination. To be noticed, the strip of the disposable device and/or the sensing module of the vital sign obtaining device may be changed depending on its purpose.

The vital sign sensing system, the vital sign obtaining device and the disposable device in accordance with the present invention may have following advantages.

1. The exterior of the elasticity arm preferably having the cavity part which corresponds with the protrusion can increase the support strength of the elasticity arm and decrease the risk of the elasticity arm damage.

2. The preferred track and the preferred hook disposed on the disposable device can prevent the movable fastener from uninstalling by the users so as that the elasticity stopper converted to the flexion state again. In addition, it can prevent the movable fastener from falling from the disposable device so as to avoid the users mistaking the malfunction of the disposable device.

3. The indicative symbol employed in one embodiment of the present invention which designed on the elasticity stopper has been showed when the disposable device had been used. The users can easily notice the indicative symbol from the outside of the disposable device that it can not be reused.

More exemplary embodiments and advantages of the present invention may be ascertained by reviewing the present disclosure and the accompanying drawing. It is intended that the description and embodiments with reference to the accompanying drawing to be considered as exemplary only.

Other embodiments of the invention will appear to those skilled in the art from consideration of the specification and practice of the invention disclosed herein. It is intended that the specification and examples to be considered as exemplary only, with a true scope and spirit of the invention being indicated by the following claims.

What is claimed is:

1. A vital sign sensing system comprising:
a disposable device comprising:
a first assembly;
an elasticity stopper formed on the first assembly and being in a flexion state under a limit and in an expansion state in nature;
a movable fastener movably assembled on said elasticity stopper and used to limit said elasticity stopper in the flexion state; and
a detective region disposed on the first assembly; and
a vital sign obtaining device comprising:
a second assembly;
a sensing module disposed on said second assembly and obtained a vital sign from the detective region; and
at least one protrusion formed on said second assembly,
wherein said movable fastener is uninstalled from said elasticity stopper by said protrusion during said first assembly assembling to said second assembly in a first time, and said elasticity stopper is converting from the flexion state to the expansion state to prevent said first assembly from being reused with said second assembly.

2. The vital sign sensing system as claimed in claim 1, wherein said elasticity stopper comprises:
at least one elasticity arm extending outer from said first assembly and having a cavity part formed on an exterior of the elasticity arm, wherein said cavity part clasps the protrusion when the elasticity stopper is in an expansion state to prevent said first assembly from being reused with said second assembly.

3. The vital sign sensing system as claimed in claim 1, wherein said disposable device further comprises:
a hook formed in said first assembly; and
a track formed in said first assembly between said hook and said elasticity stopper;
wherein said movable fastener is uninstalled from said elasticity stopper to moving along with said track and clasping said hook for each other during said first assembly assembling to said second assembly in the first time.

4. The vital sign sensing system as claimed in claim 1, wherein said elasticity stopper further comprises an indicative symbol, when said movable fastener is assembled to said elasticity stopper, said indicative symbol has been hidden, and when said movable fastener is uninstalled from said elasticity stopper, said indicative symbol has been showed.

5. A disposable device, comprising:
a first assembly;
an elasticity stopper formed on said first assembly and being in a flexion state under a limit and in an expansion state in nature;
a movable fastener assembled on said elasticity stopper and used to limit said elasticity stopper in the flexion state; and
a detective region disposed on said first assembly;
wherein said disposable device is adapted to a vital sign obtaining device which comprises a second assembly, a sensing module and at least one protrusion, wherein said sensing module is disposed on said second assembly and is obtained a vital sign from the detective region, and said protrusion is formed in said second assembly, wherein said movable fastener is uninstalled from said elasticity stopper by said protrusion during said first assembly assembling to said second assembly in a first time, and said elasticity stopper is converting from the flexion state to the expansion state to prevent said first assembly from being reused with said second assembly.

6. A vital sign obtaining device adapted to a disposable device which comprises a first assembly and a detective region, and said vital sign obtaining device comprising:
a second assembly;
a sensing module disposed on said second assembly and obtained vital sign from said detective region; and
at least one protrusion formed on said second assembly,
wherein said disposable device further comprises an elasticity stopper and a movable fastener, said elasticity stopper is formed on said first assembly and is in a flexion state when said movable fastener assembled on said elasticity stopper and in an expansion state when said movable fastener is uninstalled from said elasticity stopper by said protrusion during said first assembly assembling to said second assembly in the first time to prevent said first assembly being reused with said second assembly.
